(19) United States
(12) Patent Application Publication
(10) Pub. No.: US 2013/0150741 A1
(43) Pub. Date: Jun. 13, 2013

(54) BIO-SIGNAL TRANSFER DEVICE, VEHICLE CONTROL DEVICE, VEHICLE AUTOMATIC CONTROL SYSTEM AND METHOD USING THEREOF

(71) Applicants: Hyung Wook NOH, Daejeon (KR); YongWon Jang, Daejeon (KR); Sooyeul Lee, Daejeon (KR); Inbum Lee, Daejeon (KR); Yoon Seon Song, Daejeon (KR); Seunghwan Kim, Daejeon (KR)

(72) Inventors: Hyung Wook NOH, Daejeon (KR); YongWon Jang, Daejeon (KR); Sooyeul Lee, Daejeon (KR); Inbum Lee, Daejeon (KR); Yoon Seon Song, Daejeon (KR); Seunghwan Kim, Daejeon (KR)

(73) Assignee: Electronics and Telecommunications Research Institute, Daejeon (KR)

(21) Appl. No.: 13/567,955

(22) Filed: Aug. 6, 2012

(30) Foreign Application Priority Data
Dec. 8, 2011 (KR) 10-2011-0131103

(51) Int. Cl. A61B 5/024

(52) U.S. Cl. 600/508

(57) ABSTRACT

Disclosed is a bio-signal transfer device which includes a bio-signal measuring unit which includes at least one sensor and measures a bio-signal of a driver through the sensor; a signal processing unit which removes noise from the bio-signal and converts the noise-removed bio-signal into a digital signal; and a transmission unit which transfers the digital signal by wireless.

### CROSS-REFERENCE TO RELATED APPLICATIONS

A claim for priority under 35 U.S.C. §119 is made to Korean Patent Application No. 10-2011-0131103 filed Dec. 8, 2011, in the Korean Intellectual Property Office, the entire contents of which are hereby incorporated by reference.

### BACKGROUND

The inventive concepts described herein relate to a bio-signal transfer device, a vehicle control device, a vehicle automatic control system, and a method using the same.

Cardiac disorders such as cardiogenic shock or arrhythmia may suddenly occur without forewarning symptom. The symptom of the cardiac disorders may be various from a minor symptom such as palpitation to a serious symptom such as syncope and sudden death. An accident may occur when does not cope with such symptom. For example, if a cardiac disorder occurs during driving, a driver may be at a non-controllable state. In this case, the chance that a full-scale accident occurs may become high.

A driver may not control a driving state actively at an emergence situation due to the cardiac disorder. Thus, there is needed a control device which controls a driving state of a vehicle actively upon occurring of an emergence situation by comprehending a state of a driver.

### SUMMARY

Example embodiments of the inventive concept provide a bio-signal transfer device comprising a bio-signal measuring unit which includes at least one sensor and measures a bio-signal of a driver through the sensor; a signal processing unit which removes noise from the bio-signal and converts the noise-removed bio-signal into a digital signal; and a transmission unit which transfers the digital signal by wireless.

In example embodiments, the bio-signal measuring unit has a patch shape to be attached to a body of the driver.

In example embodiments, the bio-signal measuring unit is connected with a component of a vehicle contacting with a body of the driver.

In example embodiments, the transmission unit sends the digital signal in a Bluetooth manner.

Example embodiments of the inventive concept also provide a vehicle control device comprising a reception unit which receives a bio-signal; a bio-signal analyzing unit which analyzes the bio-signal to judge a physical condition of the driver and whether a danger situation occurs; and a control unit which controls a driving of a vehicle based on the judgment result of the bio-signal analyzing unit.

In example embodiments, the bio-signal analyzing unit comprises a noise remover which removes noise from the bio-signal through an algorithm; a heartbeat detector which detects a heartbeat from the noise-removed bio-signal; and an arrhythmia judging unit which judges occurrence of arrhythmia based on the noise-removed bio-signal and the detected heartbeat.

In example embodiments, the arrhythmia judging unit comprises an VF detector which judge ventricular fibrillation of a driver; an ST computing unit which analyzes an ST-segment to judge myocardial ischemia of a driver; and a QRS judging unit which analyzes a QRS waveform to judge arrhythmia of the driver.

In example embodiments, the vehicle control device further comprises a display unit which outputs the bio-signal and the danger situation judgment result provided from the bio-signal analyzing unit.

In example embodiments, the vehicle control device further comprises an alarm unit which outputs an alarm signal to the outside according to the bio-signal and the danger situation judgment result provided from the bio-signal analyzing unit.

In example embodiments, the alarm unit sends the danger situation judgment result to an external server by wireless.

Example embodiments of the inventive concept also provide a vehicle automatic control system comprising a bio-signal transfer device; and a vehicle control device. The bio-signal transfer device comprises a bio-signal measuring unit which includes at least one sensor and measures a bio-signal of a driver through the sensor; a signal processing unit which removes noise from the bio-signal and converts the noise-removed bio-signal into a digital signal; and a transmission unit which transfers the digital signal by wireless. The vehicle control device comprises a reception unit which receives the digital signal; a bio-signal analyzing unit which analyzes the digital signal to judge arrhythmia and judges a physical condition of the driver and whether a danger situation occurs according to the judgment result; and a control unit which controls a driving of a vehicle based on the judgment result of the bio-signal analyzing unit.

Example embodiments of the inventive concept also provide a vehicle automatic control method comprising measuring a bio-signal of a driver; sending the measured bio-signal to an analysis module by wireless; analyzing the transmitted signal to judge arrhythmia of a driver; judging a physical condition of the driver and whether a danger situation occurs, according to the judgment result; and actively controlling a driving of a vehicle when a driver is at a danger situation.

In example embodiments, the analyzing the transmitted signal to judge arrhythmia of a driver comprises judging ventricular fibrillation of a driver;

analyzing an ST-segment to judge myocardial ischemia of the driver; and analyzing a QRS waveform to judge arrhythmia of the driver.

In example embodiments, the vehicle automatic control method further comprises displaying the physical condition and the danger situation judgment result through an output device.

In example embodiments, the vehicle automatic control method further comprises transferring an alarm signal and danger situation information to the outside when the driver is at a danger situation.

### BRIEF DESCRIPTION OF THE FIGURES

The above and other objects and features will become apparent from the following description with reference to the following figures, wherein like reference numerals refer to like parts throughout the various figures unless otherwise specified, and wherein

FIG. 1 is a block diagram schematically illustrating a bio-signal transfer device according to an embodiment of the inventive concept.

FIG. 2 is a detailed block diagram illustrating a bio-signal transfer device in FIG. 1.

FIG. 3 is a block diagram schematically illustrating a vehicle control device according to an embodiment of the inventive concept.

FIG. 4 is a block diagram schematically illustrating a bio-signal analyzing unit in FIG. 3 according to an embodiment of the inventive concept.

FIG. 5 is a block diagram schematically illustrating a vehicle control device according to another embodiment of the inventive concept.

FIG. 6 is a block diagram schematically illustrating a vehicle control device according to still another embodiment of the inventive concept.

FIG. 7 is a diagram schematically illustrating a vehicle automatic control system according to an embodiment of the inventive concept.

FIG. 8 is a flowchart illustrating a vehicle automatic control method according to an embodiment of the inventive concept.

### DETAILED DESCRIPTION

Embodiments will be described in detail with reference to the accompanying drawings. The inventive concept, however, may be embodied in various different forms, and should not be construed as being limited only to the illustrated embodiments. Rather, these embodiments are provided as examples so that this disclosure will be thorough and complete, and will fully convey the concept of the inventive concept to those skilled in the art. Accordingly, known processes, elements, and techniques are not described with respect to some of the embodiments of the inventive concept. Unless otherwise noted, like reference numerals denote like elements throughout the attached drawings and written description, and thus descriptions will not be repeated. In the drawings, the sizes and relative sizes of layers and regions may be exaggerated for clarity.

It will be understood that, although the terms “first”, “second”, “third”, etc., may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section discussed below could be termed a second element, component, region, layer or section without departing from the teachings of the inventive concept.

Spatially relative terms, such as “beneath”, “below”, “lower”, “under”, “above”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” or “under” other elements or features would then be oriented “above” the other elements or features. Thus, the exemplary terms “below” and “under” can encompass both an orientation of above and below. The device may be otherwise oriented (rotated 90 degrees or at other orientations) and the spatially relative descriptors used herein interpreted accordingly. In addition, it will also be understood that when a layer is referred to as being “between” two layers, it can be the only layer between the two layers, or one or more intervening layers may also be present.

The terminology used herein is for the purpose of describing particular embodiments only and is not intended to be limiting of the inventive concept. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof As used herein, the term “and/or” includes any and all combinations of one or more of the associated listed items. Also, the term “exemplary” is intended to refer to an example or illustration.

It will be understood that when an element or layer is referred to as being “on”, “connected to”, “coupled to”, or “adjacent to” another element or layer, it can be directly on, connected, coupled, or adjacent to the other element or layer, or intervening elements or layers may be present. In contrast, when an element is referred to as being “directly on,” “directly connected to”, “directly coupled to”, or “immediately adjacent to” another element or layer, there are no intervening elements or layers present.

Unless otherwise defined, all terms (including technical and scientific terms) used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of the relevant art and/or the present specification and will not be interpreted in an idealized or overly formal sense unless expressly so defined herein.

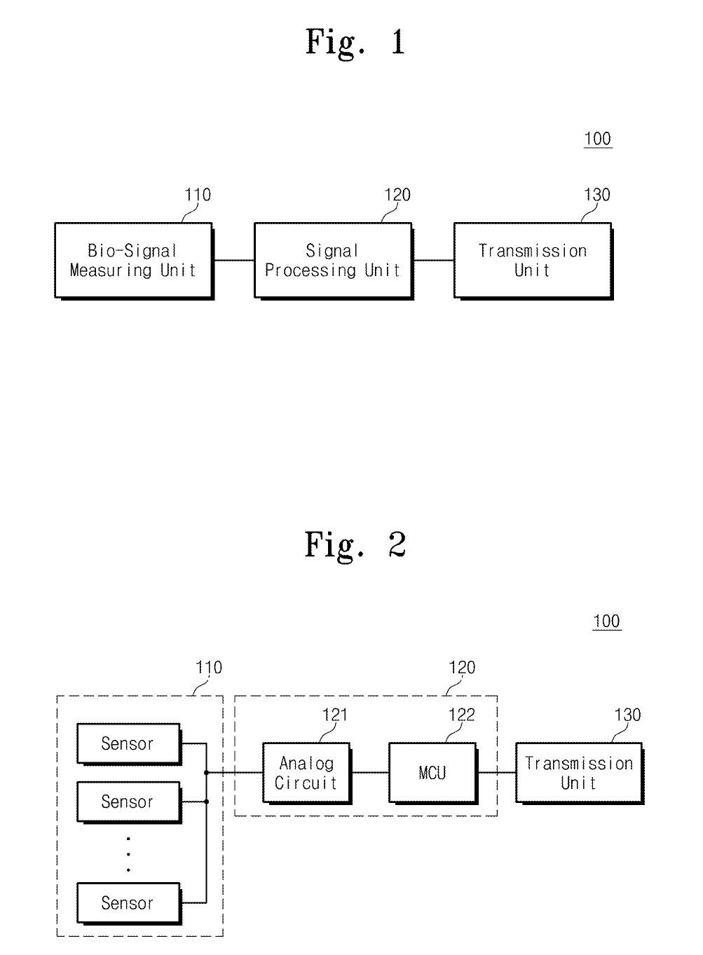

FIG. 1 is a block diagram schematically illustrating a bio-signal transfer device according to an embodiment of the inventive concept. Referring to FIG. 1, a bio-signal transfer device 100 may include a bio-signal measuring unit 110, a signal processing unit 120, and a transmission unit 130.

The bio-signal measuring unit 110 may measure a bio-signal of a driver. The bio-signal measuring unit 110 may be attached to a body of the driver independently. In this case, the bio-signal measuring unit 110 may be a patch-type unit. The bio-signal measuring unit 110 may be embedded within a component of a vehicle, for example, a handle or a seat.

A bio-signal measured by the bio-signal measuring unit 110 may be various. For example, the bio-signal may include electro-cardiogram (ECG), electro-encephalogram (EEG), electro-myogram (EMG), galvanic skin reflex (GSR), electro-oculography (EOG), Pulse plethysmography (PPG), amount of exercise on breathing rate and time, and the like. The inventive concept will be described on the basis of the electro-cardiogram (ECG). However, the inventive concept is not limited thereto.

The signal processing unit 120 may remove noise by filtering a bio-signal measured by the bio-signal measuring unit 110. The signal processing unit 120 may amplify a noise-removed bio-signal. A magnitude of a bio-signal may be very small, for example, below 1 mV. Thus, amplification of a bio-signal may be required to analyze a bio-signal. The signal processing unit 120 may convert the amplified bio-signal into a digital signal.

The transmission unit 130 may transfer a digital signal input from the signal processing unit 120 by wireless. A wireless communication manner of the transmission unit 130 may not be limited. For example, the transmission unit 130 may transfer a digital signal by a Bluetooth manner.

The bio-signal transfer device 100 according to an embodiment of the inventive concept may measure a bio-signal of a driver to remove noise of the bio-signal. Further, the bio-signal transfer device 100 may amplify the noise-removed bio-signal to convert it into a digital signal, and may transfer the digital signal. Thus, it is possible to wirelessly transfer bio information of a driver in real time.

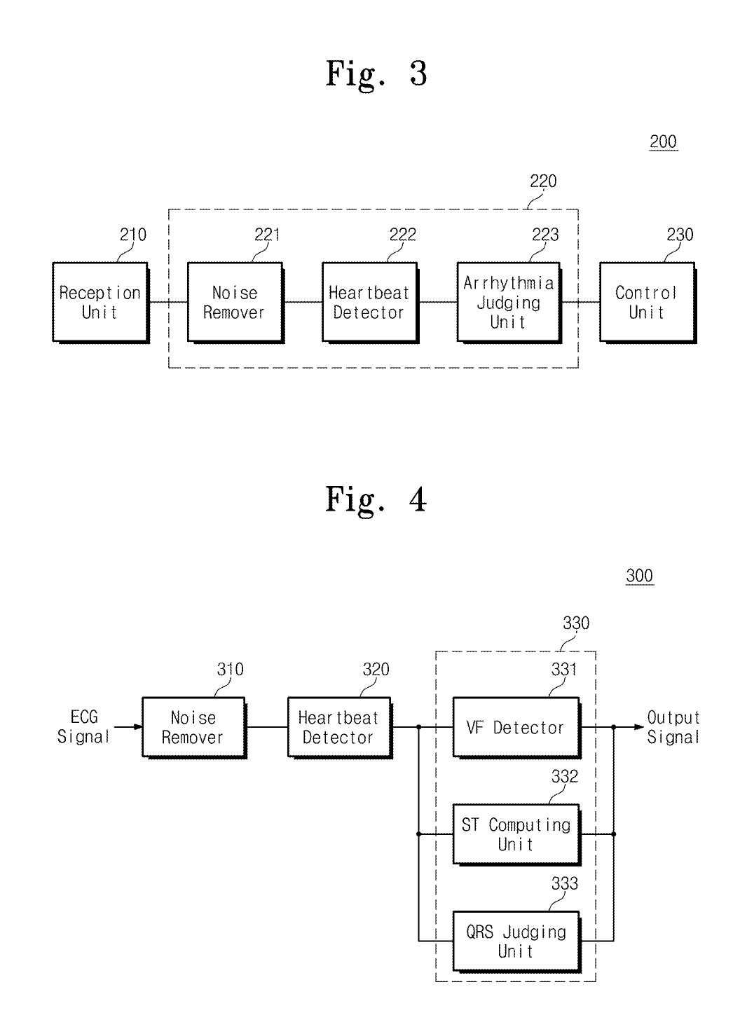

FIG. 2 is a detailed block diagram illustrating a bio-signal transfer device in FIG. 1. Referring to FIG. 2, a bio-signal transfer device 100 may include a bio-signal measuring unit 110, a signal processing unit 120, and a transmission unit 130.

The bio-signal measuring unit 110 may include at least one or more sensors. Each sensor may measure different bio-signals of a driver. Bio-signals measured by the sensors may be gathered and provided to the signal processing unit 120.

The signal processing unit 120 may include an analog circuit 121 and an MCU 122. The signal processing unit 120 may convert a bio-signal input from the bio-signal measuring unit 110 into a signal suitable for analysis, and may send it to the transmission unit 130.

The analog circuit 121 may remove noise of a bio-signal input from the bio-signal measuring unit 110 through a filter. The bio-signal may have a high noise property due to a small size. Also, since a body is organized by organic combination of organs, it is difficult to measure a signal associated with only a point. For example, a breathing signal may be measured together upon measuring of electro-cardiogram (ECG), or electro-myogram (EMG) may be measured together upon measuring of electro-encephalogram (EEG). Thus, a filter may be required to separate only a target signal from a measured signal.

The analog circuit 121 may amplify a noise-removed bio-signal. The analog circuit 121 may send the amplified bio-signal to the MCU 122.

The MCU 122 may convert a bio-signal input from the analog circuit 121 into a digital signal through an analog-to-digital converter. The MCU 122 may include a digital filter. The digital filter may remove noise that is not removed by an analog filter. The MCU 122 may output a bio-signal converted into a digital signal to the transmission unit 130. The transmission unit 130 may send a bio-signal input from the MCU 122 by wireless.

Thus, the bio-signal transfer device of the inventive concept may measure a bio-signal to convert and send the measured bio-signal into a signal suitable for analysis.

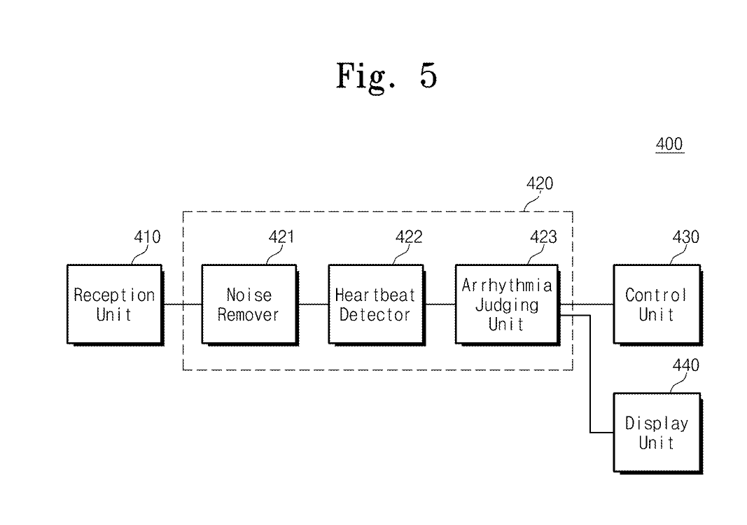

FIG. 3 is a block diagram schematically illustrating a vehicle control device according to an embodiment of the inventive concept. Referring to FIG. 3, a vehicle control device 200 may include a reception unit 210, a bio-signal analyzing unit 220, and a control unit 230.

The reception unit 210 may receive a bio-signal sent from a bio-signal transfer device. The reception unit 210 may transfer the input bio-signal to the bio-signal analyzing unit 220.

The bio-signal analyzing unit 220 may judge physical condition and danger situation of a user using the input bio-signal. The bio-signal analyzing unit 220 may include a noise remover 221, a heartbeat detector 222, and an arrhythmia judging unit 223.

The noise remover 221 may remove noise from a bio-signal transferred from the reception unit 210 through algorithm. For example, electro-cardiogram (ECG) may include noise generated due to external power, motion of a driver, electro-myogram (EMG), and the like. A peak of noise included in the electro-cardiogram (ECG) can be abnormally calculated as a pulse. For this reason, the noise removing algorithm may be used to detect only an electro-cardiogram (ECG) signal.

The heartbeat detector 222 may detect a heartbeat, that is, a pulse from the electro-cardiogram (ECG) signal passing through the noise remover 221. The heartbeat detected by the heartbeat detector 222 may be provided to the arrhythmia judging unit 223 together with electro-cardiogram.

The arrhythmia judging unit 223 may judge a danger situation of a driver, for example, whether arrhythmia occurs, based on a heartbeat and electro-cardiogram (ECG). The arrhythmia judging unit 223 may judge an arrhythmia type when occurrence of arrhythmia is detected. Occurrence of arrhythmia may cause a cardiac disorder. The arrhythmia may include ventricular fibrillation (VF), ischemic heart failure, premature ventricular contraction (PVC), and the like. The arrhythmia judging unit 223 may provide the control unit 230 with judged results (including whether arrhythmia occurs and an arrhythmia type), based on a heartbeat and electro-cardiogram (ECG).

The control unit 230 may receive the judged results from the arrhythmia judging unit 223. In the event that the judged results indicate that a driver is at a danger situation, the control unit 230 may control a driving of a vehicle. In example embodiments, the control unit 230 may decrease a driving speed of a vehicle at a danger situation. The control unit 230 may control a driving direction of a vehicle at a danger situation.

With the above description, the vehicle control device of the inventive concept may judge a danger situation of a driver based on a bio-signal sent from a bio-signal transfer device. The vehicle control device may control a driving of a vehicle according to a judgment result. Thus, it is possible to prevent an accident due to a sudden body disorder.

FIG. 4 is a block diagram schematically illustrating a bio-signal analyzing unit in FIG. 3 according to an embodiment of the inventive concept. Referring to FIG. 4, a bio-signal analyzing unit 300 may include a noise remover 310, a heartbeat detector 320, and an arrhythmia judging unit 330.

The noise remover 310 may remove noise from a bio-signal transferred from a reception unit 210. The noise remover 310 may remove a signal not associated with a pulse, so that a heartbeat is detected more exactly.

The heartbeat detector 320 may detect a heartbeat from a bio-signal noise of which is removed by the noise remover 310. A heartbeat per time may vary according to a physical state of a body. Thus, sharp variation of a heartbeat may mean that the chance that there is something wrong become high. The heartbeat may be used as an index to judge an abnormal situation of a driver. However, the heartbeat may be varied due to other factors (e.g., stress, motion, etc.) as well as a cardiac disorder. An arrhythmia detecting operation may be required to diagnose an abnormal situation more exactly using only a heartbeat.

The arrhythmia judging unit 330 may judge whether arrhythmia occurs, according to a heartbeat and electro-cardiogram (ECG). When arrhythmia occurs, the arrhythmia judging unit 330 may judge an arrhythmia type. The arrhythmia judging unit 330 may include a ventricular fibrillation (VF) detector 331, an ST computing unit 332, and a QRS judging unit 333.

The VF detector 331 may judge ventricular fibrillation (VF) of a driver. The ventricular fibrillation (VF) may be a type of arrhythmia, and may be a state that a contraction of a heart is not made normally due to electric stimulations of a heart at various points. A blood may not be ejected from a heart at the ventricular fibrillation (VF) state. When an action corresponding to the ventricular fibrillation (VF) state is not taken, a person may die within several minutes due to circulatory failure. The VF detector 331 may judge a ventricular fibrillation (VF) state of a driver based on input electro-cardiogram (ECG).

The ST computing unit 332 may analyze ST-segment to judge myocardial ischemia of a driver. Herein, the myocardial ischemia may be an imbalance between myocardial oxygen supply and demand. Left untreated, it may result in angina pectoris, myocardial stunning, myocardial hibernation, ischemic preconditioning, postconditioning, or under the most severe instances, acute coronary syndrome and myocardial infarction. Myocardial ischemia may be the pathological state underlying ischaemic heart disease.

The ST-segment may be a segment indicating an initial portion of ventricular repolarization at electro-cardiogram (ECG). A heart state may be diagnosed by analyzing a period and an amplitude variation of the ST-segment. In particular, in the event that the ST-segment indicates ischemic ST-segment depression, the chance that myocardial ischemia occurs becomes high. The ST computing unit 332 may judge myocardial ischemia of a driver based on the ST-segment of the input electro-cardiogram (ECG).

The QRS judging unit 333 may judge arrhythmia by analyzing and classifying a QRS waveform. The QRS waveform may be a part of electro-cardiogram (ECG) indicating a depolarization of the ventricle. It is possible to diagnose arrhythmia of the ventricle, for example, heart failure. The QRS judging unit 333 may judge arrhythmia of a driver and its type, based on the QRS waveform of the input electro-cardiogram (ECG).

The arrhythmia judging unit 330 may output a result obtained by combing the VF detector 331, the ST computing unit 332, and the QRS judging unit 333.

As described above, the bio-signal analyzing unit 300 according to an embodiment of the inventive concept may judge an arrhythmia state and its type by analyzing a waveform of an electro-cardiogram signal through a specific algorithm. Thus, the bio-signal analyzing unit 300 may collect accurate information associated with a danger situation to quickly cope with a danger situation.

FIG. 5 is a block diagram schematically illustrating a vehicle control device according to another embodiment of the inventive concept. Referring to FIG.

5, a vehicle control device 400 may include a reception unit 410, a bio-signal analyzing unit 420, a control unit 430, and a display unit 440. The vehicle control device 400 in FIG. 5 may be analogous to that in FIG. 3 except that the display unit 440 is added, and description thereof is thus omitted.

The display unit 440 may output a bio-signal and a danger situation judging result input from the bio-signal analyzing unit 420. The display unit 440 may include an image device and an audio device. For example, the display unit 440 may include a liquid crystal screen or a speaker. In example embodiments, the display unit 440 may show a bio-signal and a danger situation judging result through a liquid crystal screen in real time. Also, the display unit 440 may generate an alarm notifying an abnormal state such as arrhythmia.

As described above, the vehicle control device may output a physical condition and a danger situation of a driver through an output device such as an image device or an audio device such that the driver checks the physical condition and the danger situation in real time. Thus, the vehicle control device may immediately cope with the danger situation.

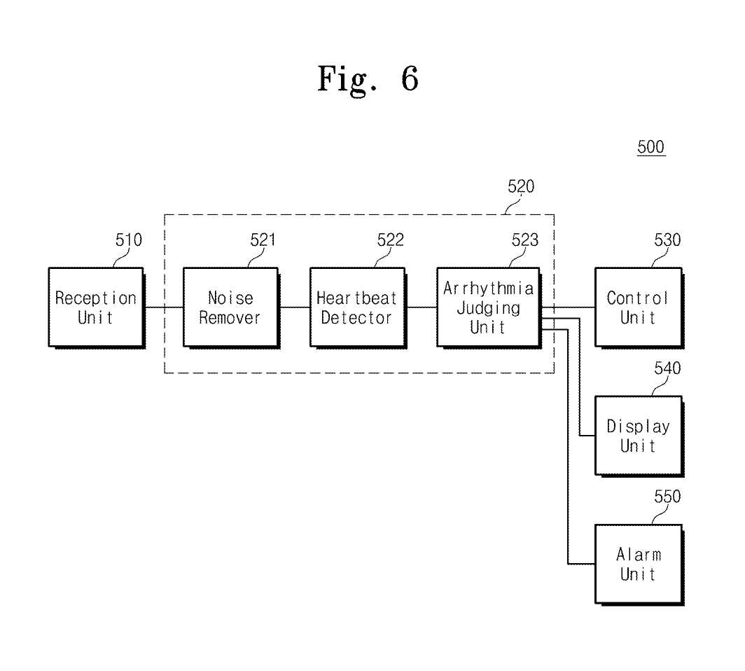

FIG. 6 is a block diagram schematically illustrating a vehicle control device according to still another embodiment of the inventive concept. Referring to FIG. 6, a vehicle control device 500 may include a reception unit 510, a bio-signal analyzing unit 520, a control unit 530, a display unit 540, and an alarm unit 550. The vehicle control device 500 in FIG. 6 may be analogous to that in FIG. 5 except that the alarm unit 550 is added, and description thereof is thus omitted.

The alarm unit 550 may output an alarm signal to the outside according to a bio-signal and a danger situation judging result input from the bio-signal analyzing unit 520. In example embodiments, upon a danger situation, the alarm unit 550 may wirelessly transfer information associated with generation of a danger situation and its type to an external server or a terminal. Also, upon a danger situation, the alarm unit 550 may send an alarm signal to a control device of a vehicle to display a danger situation. For example, an emergency light may be turned on according to the control of the alarm unit 550.

As described above, the vehicle control device 500 in FIG. 6 may immediately alarm a danger situation of a driver and send information to the outside. Thus, the vehicle control device 500 may cope with a danger situation of a driver effectively and immediately.

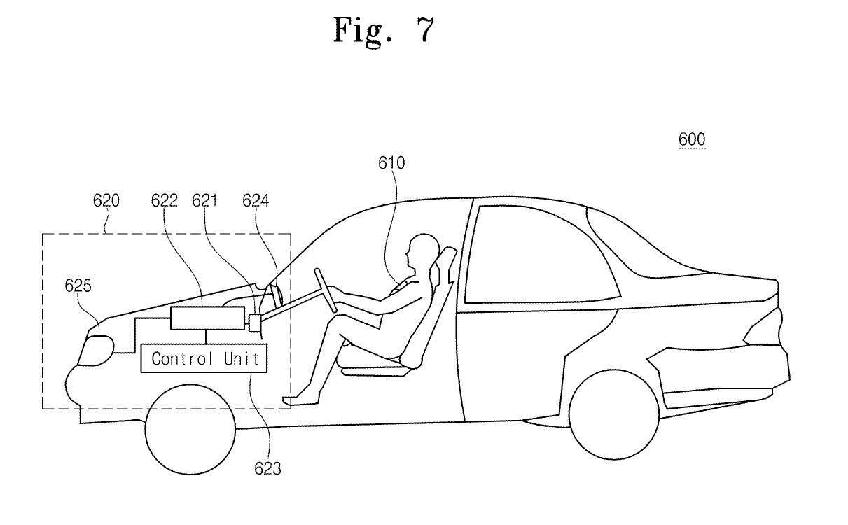

FIG. 7 is a diagram schematically illustrating a vehicle automatic control system according to an embodiment of the inventive concept. Referring to FIG. 7, a vehicle automatic control system 600 may include a bio-signal transfer device 610 and a vehicle control device 620.

The bio-signal transfer device 610 may be attached to a body of a driver independently. The bio-signal transfer device 610 may be embedded in a component of a vehicle, for example, a handle or a seat. The bio-signal transfer device 610 may measure a bio-signal to convert it into a signal suitable for analysis.

The vehicle control device 620 may include a reception unit 621, a bio-signal analyzing unit 622, a control unit 623, a display unit 624, and an alarm unit 625. The vehicle control device 620 may judge a danger situation of a driver based on a bio-signal transmitted from the bio-signal transfer device 610. The vehicle control device 620 may output a measured bio-signal and a judgment result through an output device. The vehicle control device 620 may control a driving of a vehicle according to the judgment result. Upon a danger situation, the vehicle control device 620 may display an alarm to the outside of the vehicle to send associated information to the outside.

As described above, the vehicle automatic control system 600 may measure a bio-signal of a driver to judge a danger situation. The vehicle automatic control system 600 may actively control a vehicle according to the judgment result to send a danger signal to the outside.

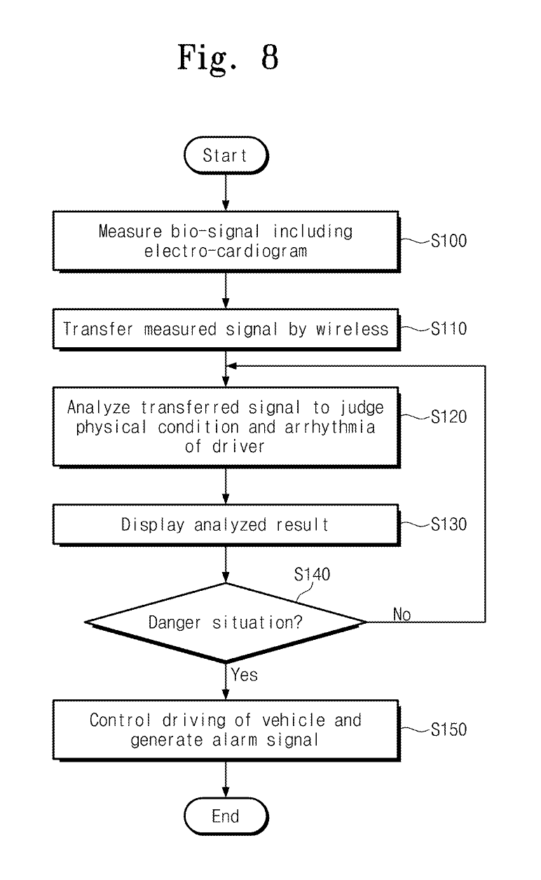

FIG. 8 is a flowchart illustrating a vehicle automatic control method according to an embodiment of the inventive concept. Referring to FIG. 8, in operation S100, a bio-signal including electro-cardiogram of a driver may be measured. In operation S110, the measured signal may be converted into a signal suitable for analysis, and the converted bio-signal may be sent to an analysis module by wireless. In operation S120, a physical condition and a danger situation of a driver, for example, occurrence of arrhythmia may be judged through analyzing of the input signal. In operation S130, the analyzed result may be displayed through an output device. In operation S140, there may be judged whether a driver is at a danger situation. If so, in operation S150, a driving of a vehicle may be actively controlled, and an alarm signal and danger situation information may be transmitted to the outside. If not, the method proceeds to operation S120 such that information of the driver may continue to be measured.

The inventive concept may be modified or changed variously. For example, a bio-signal measuring unit, a signal processing unit, and a transmission unit may be changed or modified variously according to environment and use.

While the inventive concept has been described with reference to exemplary embodiments, it will be apparent to those skilled in the art that various changes and modifications may be made without departing from the spirit and scope of the present invention. Therefore, it should be understood that the above embodiments are not limiting, but illustrative.

What is claimed is:

1. A bio-signal transfer device comprising:
a bio-signal measuring unit which includes at least one sensor and measures a bio-signal of a driver through the sensor;
a signal processing unit which removes noise from the bio-signal and converts the noise-removed bio-signal into a digital signal; and
a transmission unit which transfers the digital signal by wireless.

2. The bio-signal transfer device of claim 1, wherein the bio-signal measuring unit has a patch shape to be attached to a body of the driver.

3. The bio-signal transfer device of claim 1, wherein the bio-signal measuring unit is connected with a component of a vehicle contacting with a body of the driver.

4. The bio-signal transfer device of claim 1, wherein the transmission unit sends the digital signal in a Bluetooth manner.

5. A vehicle control device comprising:
a reception unit which receives a bio-signal;
a bio-signal analyzing unit which analyzes the bio-signal to judge a physical condition of the driver and whether a danger situation occurs; and
a control unit which controls a driving of a vehicle based on the judgment result of the bio-signal analyzing unit.

6. The vehicle control device of claim 5, wherein the bio-signal analyzing unit comprises:
a noise remover which removes noise from the bio-signal through an algorithm;
a heartbeat detector which detects a heartbeat from the noise-removed bio-signal; and
an arrhythmia judging unit which judges occurrence of arrhythmia based on the noise-removed bio-signal and the detected heartbeat.

7. The vehicle control device of claim 6, wherein the arrhythmia judging unit comprises:
an VF detector which judge ventricular fibrillation of a driver;
an ST computing unit which analyzes an ST-segment to judge myocardial ischemia of a driver; and
a QRS judging unit which analyzes a QRS waveform to judge arrhythmia of the driver.

8. The vehicle control device of claim 5, further comprising:
a display unit which outputs the bio-signal and the danger situation judgment result provided from the bio-signal analyzing unit.

9. The vehicle control device of claim 8, further comprising:
an alarm unit which outputs an alarm signal to the outside according to the bio-signal and the danger situation judgment result provided from the bio-signal analyzing unit.

10. The vehicle control device of claim 9, wherein the alarm unit sends the danger situation judgment result to an external server by wireless.

11. A vehicle automatic control system comprising:
a bio-signal transfer device; and
a vehicle control device,
wherein the bio-signal transfer device comprises:
a bio-signal measuring unit which includes at least one sensor and measures a bio-signal of a driver through the sensor;
a signal processing unit which removes noise from the bio-signal and converts the noise-removed bio-signal into a digital signal; and
a transmission unit which transfers the digital signal by wireless; and
wherein the vehicle control device comprises:
a reception unit which receives the digital signal;
a bio-signal analyzing unit which analyzes the digital signal to judge arrhythmia and judges a physical condition of the driver and whether a danger situation occurs according to the judgment result; and
a control unit which controls a driving of a vehicle based on the judgment result of the bio-signal analyzing unit.

12. A vehicle automatic control method comprising:
measuring a bio-signal of a driver;
sending the measured bio-signal to an analysis module by wireless;
analyzing the transmitted signal to judge arrhythmia of a driver;
judging a physical condition of the driver and whether a danger situation occurs, according to the judgment result; and
actively controlling a driving of a vehicle when a driver is at a danger situation.

13. The vehicle automatic control method of claim 12, wherein the analyzing the transmitted signal to judge arrhythmia of a driver comprises:
judging ventricular fibrillation of a driver;
analyzing an ST-segment to judge myocardial ischemia of the driver; and
analyzing a QRS waveform to judge arrhythmia of the driver.

14. The vehicle automatic control method of claim 12, further comprising:
displaying the physical condition and the danger situation judgment result through an output device.

15. The vehicle automatic control method of claim 12, further comprising:
transferring an alarm signal and danger situation information to the outside when the driver is at a danger situation.
